(19) United States
(12) Patent Application Publication
(10) Pub. No.: US 2004/0242994 A1
(43) Pub. Date: Dec. 2, 2004

(54) Dynamic contrast enhanced magnetic resonance imaging

(76) Inventors: John Michael Brady, Oxford (GB); Paul Anthony Armitage, Edinburgh (GB); Christian Peter Behrenbruch, Oxford (GB)

Correspondence Address: Kalow & Springut, 488 Madison Avenue, New York, NY, 10022, US

(21) Appl. No.: 10/483,705

(22) PCT Filed: Jul. 5, 2002
(86) PCT No.: PCT/GB02/03101

(30) Foreign Application Priority Data
Jul. 13, 2001 (GB) 0117187.5

(51) Int. Cl. A61B 5/05; G01V 3/00

(52) U.S. Cl. 600/420; 324/309

(57) ABSTRACT

A method of dynamic contrast enhanced magnetic resonance imaging, and of processing the signals from such imaging, in order to improve the characterisation of tissue types being imaged. A calculation of the longitudinal relaxation time T₁ is made for each voxel in the image by applying pulse sequences having different flip angles or TRs and fitting the resulting resonance signals to a model of the imaging process. Dynamic, contrast-enhanced imaging is then conducted and by using the T₁ values the results may be fitted to a pharmacokinetic model of the uptake of contrast agent in the tissue being imaged. This gives values for physiological parameters relating to the permeability of the tissue and the extravascular extracellular space volume fraction. These, together with the T₁ value provide an excellent characterisation of the tissue as malignant or benign. The parameters may be displayed using a vector map or by displaying each of them in a different colour, allowing a quick and meaningful of the image to be made.

The present invention relates to magnetic resonance imaging, and in particular to the derivation from magnetic resonance images of parameters relating to the physiology of the tissue being imaged.

Magnetic resonance imaging (MRI) techniques are widely used to image soft tissue within human (or animal) bodies and there is much work in developing techniques to analyse the resonance signals in a way which characterises the tissue being imaged, for instance as normal or diseased. However, to date conventional MRI has not been capable of distinguishing between healthy and malignant tissue. Tumours have a number of distinguishing characteristics. For example, to sustain their aggressive growth they generate millions of tiny “microvessels” that increase the local blood supply around the tumour to sustain its abnormal growth. A technique which is based on this physiology is dynamic contrast-enhanced magnetic resonance imaging (CE-MRI) and a common application of CE-MRI is for breast cancer imaging, in particular for younger women and for those cases in which a diagnosis based on x-ray mammography is inconclusive. Dynamic CE-MRI involves the intravenous injection of a contrast agent (such as gadopentetate dimeglumine Gd-DTPA) immediately prior to acquiring a set of magnetic resonance volumes or data sets, typically one a minute for several minutes. The presence of contrast agent within an imaging voxel (volume-pixel—the smallest volume element of the sample which is resolved in the image), results in an increased resonance signal. The dynamic/temporal change in the signal as the contrast agent is taken-up by the tissue and then flushed out can be observed over the time course of the experiment. Different tissue types have different contrast agent uptake and flush properties, and so study of the resonance signal over time enables identification of the different tissue types.

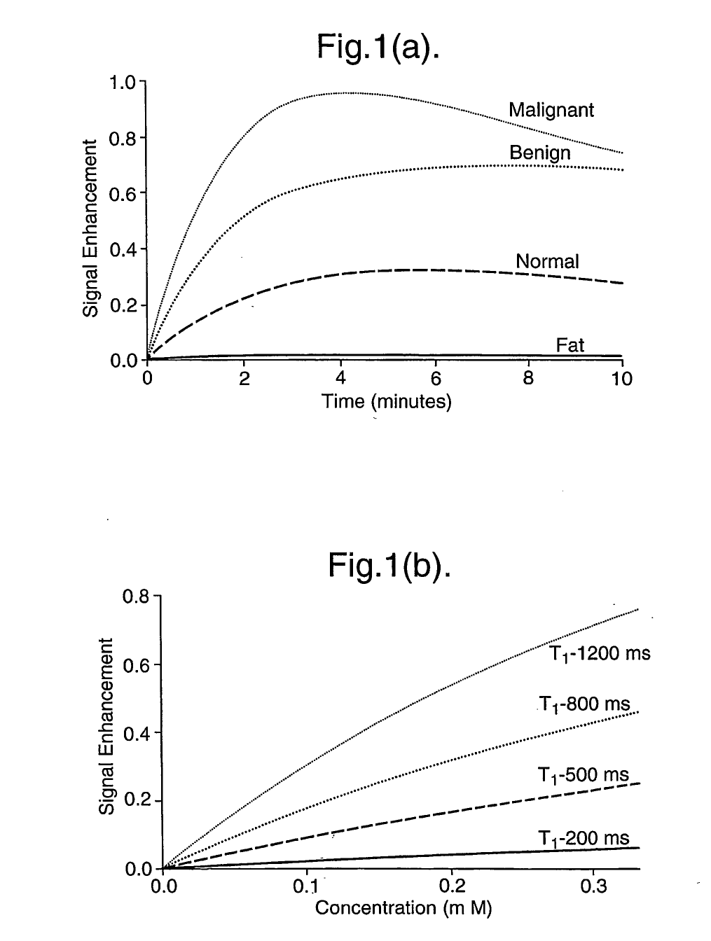

Typically, cancerous tissue shows a high and fast uptake because of the microvasculature which is leaky, while normal and fatty tissues show little contrast agent uptake. FIG. 1(a) of the accompanying drawings illustrates typical contrast agent uptake curves plotted for different tissue types. FIG. 1(b) plots signal enhancement (which is the ratio of the signal intensity after injection of contrast agent to the signal intensity obtained with no contrast agent injection) as a function of contrast agent concentration. It can be seen that malignant tissue (a tumour) is characterised by a sharp rise and overall higher enhancement than benign, normal or fatty tissue. These uptake curves have often been fitted using a pharmacokinetic model (a model which mathematically relates to the concentration of contrast agent in the tissue as a function of time with various physiological parameters of the tissue) in an attempt to give a physiologically relevant parameterisation of the curve. Study of these curves/parameters has been proposed as a technique which could identify and characterise tumours into malignant or benign classes. However, the results are currently insufficiently reliable to provide a conclusive diagnosis. One reason for this is that the pharmacokinetic model requires knowledge of the change in amount or concentration of contrast agent in the tissue over time. But the signal enhancement seen in the magnetic resonance image is not simply related to the amount of contrast agent in the tissue. Instead the relationship between the signal enhancement and the concentration of contrast agent in the sample is both non-linear, and highly dependent on the intrinsic longitudinal relaxation time (T₁ value) of the sample. The T₁ value varies greatly for different types of tissue, for instance from about 175 ms for fat, 765 ms for fibrocystic tissue, 800 ms for parenchymal tissue, 900 ms for malignant tissue and 1000 ms for a fibroadenoma (all measured at 1.0T). The variation in signal enhancement with concentration for different values for T₁ is illustrated in FIG. 1(b). The non-linearity, and also the high dependence on T₁ can be seen easily. An example of the problem this creates is that if one considers a particular voxel which is showing a high enhancement, one cannot tell whether this is due to the uptake of contrast agent or the intrinsic T₁ value of the tissue. Thus one cannot tell whether it is a physiologically-based effect (high uptake of contrast agent) or an intrinsic effect (because of the T₁ value of that type of tissue).

The present invention is concerned with a method of magnetic resonance imaging, and of MR image analysis, which enables an improved characterisation of the physiology of the sample being imaged. Further, it is concerned with the calculation and the display of physiologically meaningful parameters which allow this characterisation of the sample.

The first aspect of the invention provides a method of enhancing a dynamic contrast-enhanced magnetic resonance image comprising the steps of:

for each voxel of the image fitting to the magnetic resonance signal a parameterised pharmaco-kinetic model of the contrast enhancement process in the sample being imaged to calculate the values of parameters of the model which represent properties of the imaged sample,

and displaying the image with each of said parameters being represented in a visually distinguishable manner.

The parameters may each be represented by a different colour whose intensity is representative of the value of the parameter, or the parameters for each of a plurality of regions of the sample may be represented as components of a vector displayed for each region. At least one of the parameters may be represented by the intensity or colour of the displayed vector. Alternatively the parameters may be represented in a relative phase coherence map.

This technique contrasts with the common use of “false colour” in medical imaging. False colour is often applied to images to improve the visibility of imaged features compared with a standard grey scale image. However, with the present invention different parameters of the model which represent real properties of the imaged sample are calculated, and it is these parameters which are displayed in a visually distinguishable manner, e.g in different colours. These parameters may, for instance, represent the physiology or structure of the imaged sample, examples being the extravascular extracellular space (EES) volume fraction νₑ, the permeability surface area product per unit volume of the sample K^{trans}, the rate constant kₑₚ which is equal to K^{trans}/νₑ, or the longitudinal relaxation time (T₁) itself.

The parameterised pharmacokinetic model may be one of the known two- or three-compartment models in which the different compartments represent the blood plasma and extravascular extracellular space, and in the three-compartment model the extracellular space (whole body), and the concentration in each compartment can be expressed as a function of the initial amount of contrast agent injected, transfer coefficients between the different compartments and transfer out of the body through the kidneys. Because a tumour typically has a leaky microvasculature around it, it can be characterised by the value of the transfer constants in the model such as the EES volume fraction and the K^{trans}.

Another aspect of the invention provides a method of magnetic resonance imaging comprising the steps of:

acquiring resonance signals by applying to a subject successive electromagnetic pulse sequences, each sequence differing in a selected acquisition parameter,

and calculating from the resonance signals the longitudinal relaxation time (T₁) for the sample.

The selected acquisition parameter which differs from sequence to sequence may be the flip angle or the repetition time (TR).

It was noted above that a problem with prior art approaches is that the T₁ value affects the signal enhancement, so that regions with a high T₁ value enhance greatly even with a small uptake of contrast agent. This makes them confusingly similar in the image to (malignant) regions which enhance greatly because of a high takeup of contrast agent. This aspect of the invention provides a way of measuring the intrinsic T₁ value of the sample. This allows not only a (provisional) characterisation of the tissue type of the sample by its T₁ value, but also allows a more accurate calculation from the resonance signal of the concentration of contrast agent in the sample before application of a pharmacokinetic model, in turn allowing the more accurate calculation of the physiological parameters (such as the transfer constants) in the model.

The different flip angles or repetition time in the successive sequences may be selected to minimise the error in the T₁ value over the range of T₁ expected in the sample. One of the sequences may be the conventional initial non-contrast enhanced sequence used in CE-MRI, with one or more earlier sequences being applied each with a different flip angle or repetition time.

In one embodiment the same pulse sequence is used in three acquisitions with different acquisition parameters. However different numbers of acquisitions can be used, in which case the optimum acquisition parameters for minimising the error in the T₁ value would be different.

In one embodiment the pulse sequence is a gradient echo sequence such as a T₁ weighted 3-D fast spoiled gradient echo sequence, but other sequences such as spin echo could be used with an appropriate signal model.

The longitudinal relaxation time (the T₁ value) may be calculated by fitting the resonance signals for the different flip angles or TRs to one of the known published models of the sample's response to the pulse sequence. Such models are available which include correction for non-uniform excitation across the sample (in which case the flip angle varies to some extent across the sample), and which correct for B₁ inhomogeneity across the sample.

The method preferably gives a T₁ value for each voxel of the sample and the invention is particularly applicable to samples such as the soft tissues of the human or animal body, and in particular in the field of medical imaging to the human breast, or other soft tissues such as the prostate, liver and other organs and the brain etc.

The method of calculating the T₁ value may be provided in the context of an imaging method or analysis method as discussed above, or as a stand-alone method. This aspect of the invention therefore constitutes a method of determining T₁ values for magnetic resonance data using the steps mentioned above.

The invention extends to magnetic resonance imaging apparatus which is adapted to execute the method of the invention, and also to a computer program comprising program code means for executing the method of the invention. The computer program may be embodied on a computer-readable storage medium.

The invention will be further described by way of example with reference to the accompanying drawings in which:

FIG. 1(a) and (b) illustrate typical contrast agent uptake curves for different tissue types and the relationship between magnetic resonance signal enhancement and contrast agent concentration for different T₁ values;

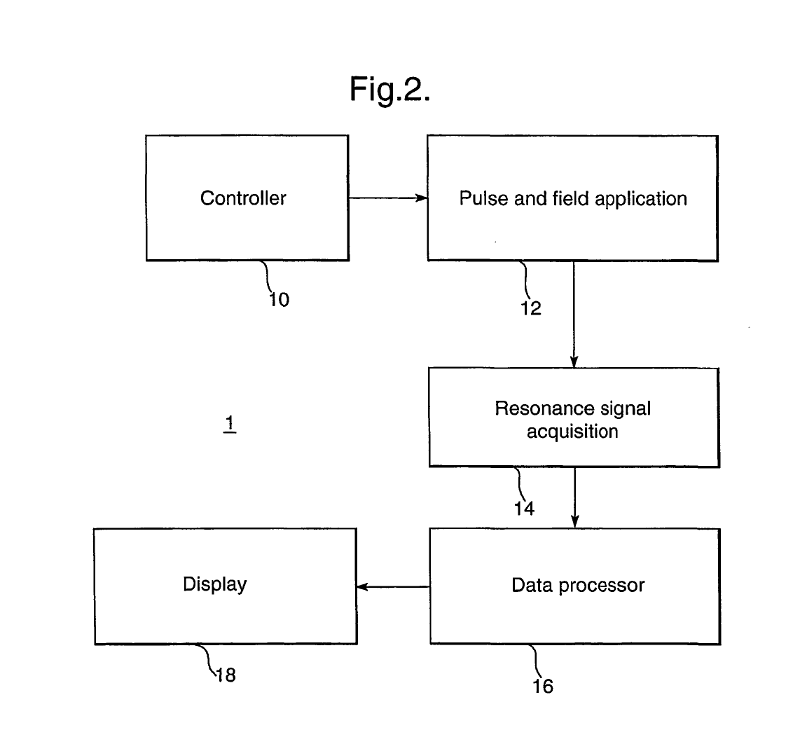

FIG. 2 schematically shows the magnetic resonance imaging apparatus and process;

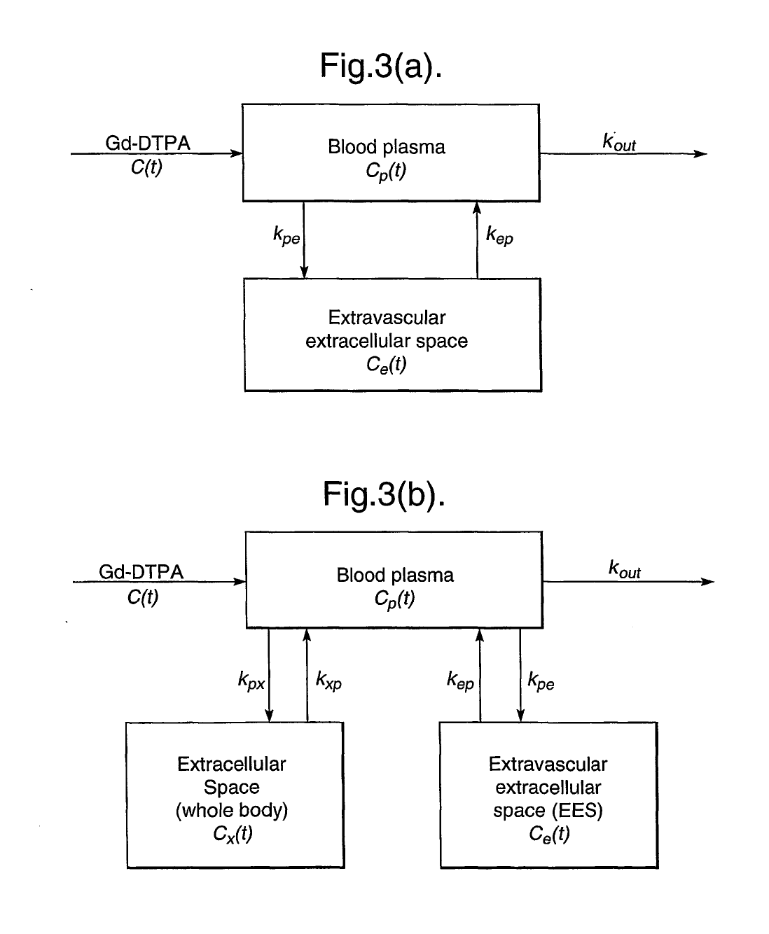

FIGS. 3A and 3B illustrate respectively two- and three-compartment pharmacokinetic models for the behaviour of contrast agent in the body;

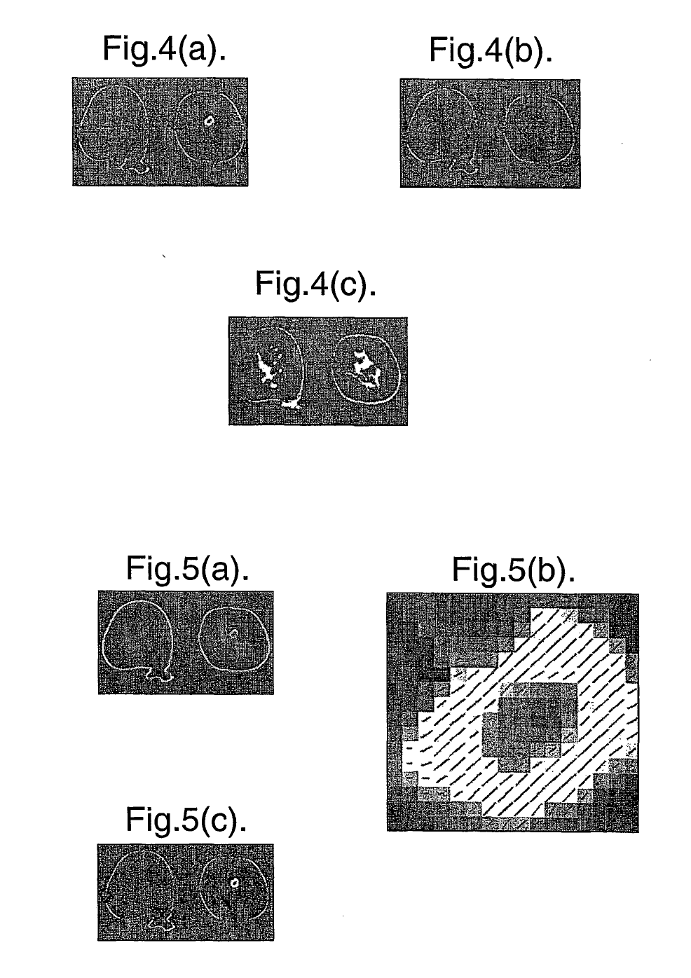

FIG. 4 illustrates pharmacokinetic parameter maps of (a) the transfer constant K^{trans}; (b) the rate constant kₑₚ; and (c) the T₁ value in a coronal breast slice containing an enhancing tumour;

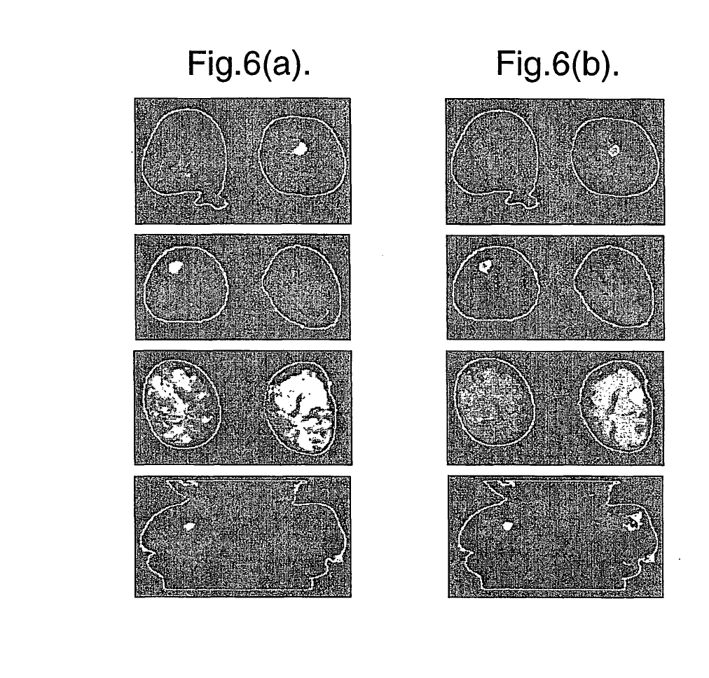

FIG. 5 illustrates displays of relevant physiological parameters using (a) the colour representation; (b) a vector overlay onto an uptake curve integral map and (c) a relative phase coherence map;

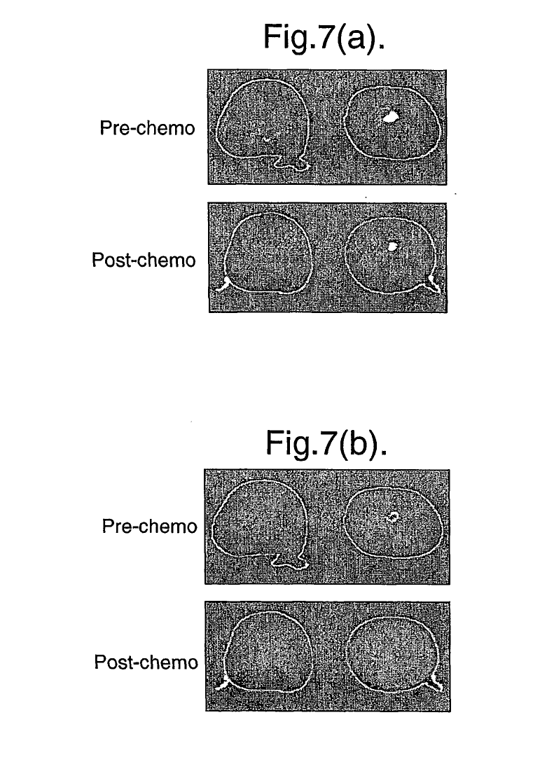

FIGS. 6(a) and (b) illustrate respectively conventional signal enhancement images and images in which the physiological parameters are calculated and displayed as different colours for four different malignant tumours; and

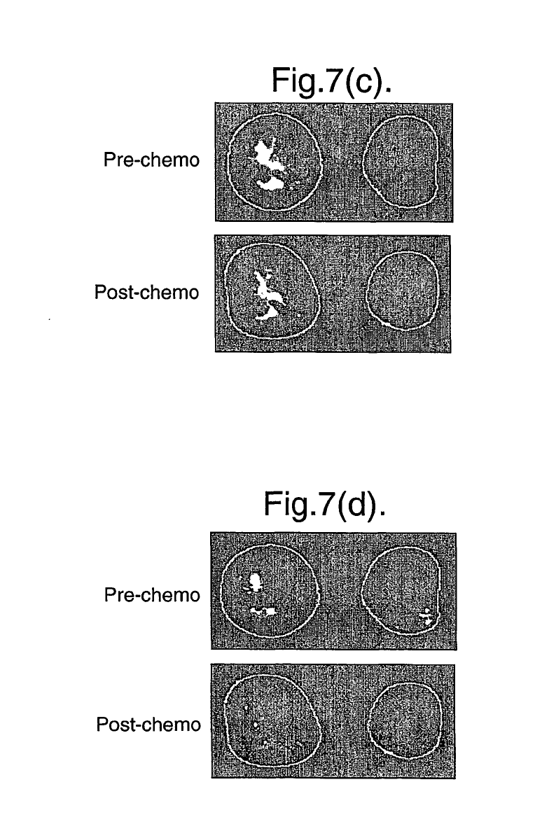

FIGS. 7(a) to (d) illustrate the pre and post chemotherapy images on two patients comparing the conventional signal enhancement technique and the physiologically based colour representation of the invention.

An embodiment of the invention will now be described with particular reference to breast imaging. FIG. 2 illustrates schematically a typical magnetic resonance imaging apparatus and process. The apparatus includes a controller 10 for allowing the user to control the apparatus 12 for applying the electromagnetic pulse sequences and magnetic fields to the sample. MRI machines typically have a number of preset pulse sequences available, though the operator is also free to vary the various sequence parameters as desired. The resonance signals are acquired at 14 and supplied to a data processor 16 which prepares the signals for display by display 18. The data processing in accordance with the present invention may be executed by the data processing facility built into the apparatus, or may be performed by a suitably programmed general purpose computer supplied with the data from the imaging apparatus.

In this embodiment the images were obtained using a GE-Signa 1.5T clinical MRI scanner using T₁-weighted 3-D fast spoiled gradient echo pulse sequence (TE/TR=4.2/8.9 ms) with an overall imaging time of around 40 seconds for a 256×256×80 volume. Clearly the invention is applicable to other magnetic resonance imaging apparatus and pulse sequences.

Firstly methods of calculating the T₁ value for each voxel of the imaged sample will be explained. This may be achieved by performing multiple acquisitions of the basic sequence, with each acquisition having either a different RF flip angle or a different TR. In the first case, TE and TR are kept constant, while for the second, TE and a are kept constant. A choice needs to be made as to which flip angles or TR values would be appropriate in order to make a reliable assessment of the transverse relaxation time, T₁₀, over the physiological range present in the normal and pathological breast. The signal obtained is highly dependent on T₁₀, TE TR and the flip angle. In a typical experiment, the ideal choice of flip angle or TR depends on the T₁₀ value and must be optimised over the physiological range of T₁₀. There are a number of analytical and numerical methods for optimising a given number of flip angles or TR values for a typical breast examination, in order that the error in T₁₀ measurement is minimised, and examples are given below for both the multiple flip angle and multiple TR approach.

The pre-contrast signal Sₙ in an FSPGR sequence is dependent upon the system gain (g), proton density (ρ), echo time (TE), flip angle (α), repetition time (TR) and the relaxation times T₁ and T*₂in the following way:

$${S}_{n} = k sin {α}_{n} \frac{1 - {ⅇ}^{- TR ( {T}_{10}^{- 1} )}}{1 - cos {α}_{n} {ⅇ}^{- TR ( {T}_{10}^{- 1} )}} \qquad [ e1 ]$$

where k=gρ exp(−TE/T*₂₀) and T₁, T₂ and T*₂ have the standard definitions. In a basic method if two acquisitions are performed with different flip angles α₁ and α₂, then the transverse relaxation time T₁₀ can be calculated from:

$${T}_{10}^{- 1} = \frac{1}{TR} ln [ \frac{{S}_{R} sin {α}_{2} cos {α}_{1} - sin {α}_{1} cos {α}_{2}}{{S}_{R} sin {α}_{2} - sin {α}_{1}} ] \qquad [ e2 ]$$

where S_{R}=S₁/S₂. However, in reality Sₙ will be corrupted by noise such that the measured signal in a voxel will be Sₙ±ΔSₙ and so the measured T₁₀ will also be in error. The aim of RF flip angle optimisation is to find the combination of n flip angles α_{1→n} which minimise the error in T₁₀, ΔT₁. If it is assumed that ΔTR=Δαₙ=0 and ΔSₙ=ΔS(∀n), simple error analysis leads to a formula for ΔT₁₀⁻¹, such that

$$\begin{matrix}{E}_{{T}_{10}^{- 1}} = \frac{Δ {T}_{10}^{- 1}}{Δ S} = {S}_{R} \frac{\partial {T}_{10}^{- 1}}{\partial {S}_{R}} \sqrt{{( \frac{1}{{S}_{1}} )}^{2}} + {( \frac{1}{{S}_{2}} )}^{2} \\ where & [ e3 ] \\ \frac{\partial {T}_{10}^{- 1}}{\partial {S}_{R}} = \frac{sin {α}_{1} sin {α}_{2} ( cos {α}_{2} - cos {α}_{1} )}{TR ( {S}_{R} sin {α}_{2} - sin {α}_{1} ) ( {S}_{R} sin {α}_{2} cos {α}_{1} - sin {α}_{1} cos {α}_{2} )} & [ e4 ]\end{matrix}$$

This error in ΔT₁₀⁻¹ can then be transposed to give the error ΔT₁₀, such that

$${E}_{{T}_{10}} = \frac{Δ {T}_{10}}{Δ S} = \frac{1}{Δ S} \frac{\partial {T}_{10}}{\partial {T}_{10}^{- 1}} Δ {T}_{10}^{- 1} = \frac{1}{Δ S} {T}_{10}^{2} Δ {T}_{10}^{- 1} = - {T}_{10}^{2} {E}_{{T}_{10}^{- 1}} \qquad [ e5 ]$$

Optimisation of two flip angles α₁ and α₂ is then achieved for a given T₁₀ and TR, by calculating the combination which gives the minimum value for E_{T}_{₁₀} or E_{T}_{₁₀}_{⁻¹}.

The above equations provide optimisation for two flip angles only, but an optimal estimation method is, in practice based on more flip angles. In this case a numerical simulation (using a Monte Carlo method) can be made using the signal model of equation e1 corrupted by random noise. The noise model can be assumed to be gaussian because for typical breast imaging studies the signal-to-noise ratio (SNR) is sufficiently high, such that the gaussian approximation is adequate. As an example of this a numerical phantom can be constructed that consists of 20 square regions of size 64×64 (4096 points per region), each of which is assigned a theoretical T₁₀ value in the range of 150-1100 ms (step size 50 ms). In each of these regions the ideal signal can be calculated from Eq. e1 for each chosen αₙ, assuming values for TR and k of 8.9 ms and 1200, respectively. This TR value corresponds to the GE FSPGR sequence and the ‘gain term’ k gives typical signal values. In reality, k is likely to vary across an image as determined by the proton density, as TE<<T*₂ and the system gain is assumed to be constant for a given image. The ideal signal Sₙ in each voxel can then be corrupted by gaussian noise of standard deviation ΔS, by adding a random component generated from the gaussian noise distribution. A noise-corrupted data set is constructed for each flip angle αₙ and Eq. e1 can be fitted to the data to obtain a value for k and T₁₀ in each voxel. The mean (μ) and standard deviation (σ) of the calculated T₁₀ can be obtained in each region (with different ideal T₁₀) and the value of σ is taken as the error E_{T}_{₁₀} in that region. This procedure can be repeated for different αₙ combinations and the optimum combination will be the one with the minimum mean E_{T}_{₁₀}, where the mean is taken as the mean error over all 20 regions. The optimum flip angles suggested from the Monte Carlo simulations are presented in Table 1 which shows optimised flip angles for n=2-5 for the case where one flip angle is fixed at 10°. The data represents the flip angle choice that minimises the standard deviation σ_{T}_{₁₀} of T₁₀ in the simulated data.

TABLE 1

| n | σ_{T} _{₁₀} | α₁ | α₂ | α₃ | α₄ | α₅ | σ_{T} _{₁₀} (opt) |
|---|---|---|---|---|---|---|---|
| 2 | 29 ms | 3° | 10° | — | — | — | 29 ms |
| 3 | 21 ms | 3° | 10° | 17° | — | — | 24 ms |
| 4 | 18 ms | 3° | 4° | 10° | 15° | — | 26 ms |
| 5 | 16 ms | 3° | 4° | 10° | 16° | 17° | 29 ms |

T₁₀ calculations and optimisation using the multiple TR approach.

The pre-contrast signal Sₘ in FSPGR sequence acquired with repetition time TRₘ, assuming a homogenous voxel, is given

$${S}_{m} = k sin α \frac{1 - {ⅇ}^{- {TR}_{m} ( {T}_{10}^{- 1} )}}{1 - cos α {ⅇ}^{- {TR}_{m} ( {T}_{10}^{- 1} )}} \qquad [ e11 ]$$

where k=g ρ exp(−TE/T*₂₀). In this example, to obtain optimum TR values for breast imaging, Monte Carlo methods are used, similar to those performed above.

A numerical phantom was constructed, as described above, whereby the ideal MR signal was simulated and corrupted with gaussian noise. However, in this case, the numerical phantom was calculated for multiple TR values (with constant k=1200 and α=10°) and the Sₘ vs. TRₘ plot fitted by e11 to obtain T₁₀ values in each voxel. As before, different combinations of TRₘ are used and the optimum found as the combination that minimises the mean error in T₁₀ over the physiological range.

The results of the simulated data are illustrated in Table 2 below which shows optimised TR values for n=2-5, obtained using Monte Carlo methods for the case where one TR is fixed at the minimum value of 8.9 ms. The data represents the TR choice (for a given n) that minimises the standard deviation of T₁₀ (σ_{T}_{₁₀}) in the simulated data. The remaining TR values were optimised for the range 10-960 ms (step size 50 ms) where TR₁≠TR₂≠TR₃≠TR₄≠TR₅. This suggests that the most reliable measurement of T₁₀ will be obtained from a two-point fit with the lowest possible (TRₘᵢₙ) and highest possible (TRₘₐₓ) TR values. In practice, the TRₘᵢₙ is fixed by the imaging sequence (8.9 ms, in this case) and TRₘₐₓ must be long enough such most of the magnetisation has recovered into the longitudinal plane, the sequence has little T₁ weighting and therefore becomes predominately weighted by proton density. The simulations suggest that TRₘₐₓ>500 ms is an appropriate criterion for this upper limit in breast imaging, as further increases in TRₘₐₓ do not lead to significant reductions in the T₁₀ measurement error (σ_{T}_{₁₀}); i.e. {TR₁, TR₂}={8.9 ms, 960 ms) gives σ_{T}_{₁₀}=17.6 ms, while {TR₁, TR₂}={8.9 ms, 510 ms) gives σ_{T}_{₁₀}=18.3 ms. Obviously, this TR limit will only be applicable for the sequence parameters used here (i.e. with α=10°) and for larger flip angles, it is likely that a longer TR will be necessary.

TABLE 2

| n | σ_{T} _{₁₀} | TR₁ | TR₂ | TR₃ | TR₄ | TR₅ |
|---|---|---|---|---|---|---|
| 2 | 18 ms | 8.9 ms | 960 ms | — | — | — |
| 3 | 13 ms | 8.9 ms | 10 ms | 960 ms | — | — |
| 4 | 12 ms | 8.9 ms | 10 ms | 910 ms | 960 ms | — |
| 5 | 12 ms | 8.9 ms | 10 ms | 60 ms | 910 ms | 960 ms |

In the above it has been assumed that the whole slice across the sample has the same flip angle and that the bias field B₁ is homogeneous across the sample. In practice neither of these assumptions is correct, but the signal can be corrected using known techniques.

Having obtained the T₁ values for each of the voxels in the image, it is then possible in accordance with the invention to use these in an improved analysis of the resonance signals based on a pharmacokinetic model. An embodiment of this process will now be described.

The relationship between the signal in a gradient echo sequence, such as FSPGR, and the Gd-DTPA concentration is given by equation e6 below,

$$S ( {C}_{t} ) = g {ρⅇ}^{- TE ( {T}_{20}^{* - 1} + {R}_{2} {C}_{t} )} sin α \frac{1 - {ⅇ}^{- TR ( {T}_{10}^{- 1} + {R}_{1} {C}_{t} )}}{1 - cos α {ⅇ}^{- TR ( {T}_{10}^{- 1} + {R}_{1} {C}_{t} )}} \qquad [ e6 ]$$

where T*₂₀ and T₁₀, are the T*₂ and T₁ values before injection of Gd-DTPA and R₁ and R₂ are the tissue relaxation rates for Gd-DTPA, defined by

$$\frac{1}{{T}_{1}} = \frac{1}{{T}_{10}} + {R}_{1} {C}_{t} \frac{1}{{T}_{2}} = \frac{1}{{T}_{20}} + {R}_{2} {C}_{t}$$

The values for R₁ and R₂have been found by experiment to be R₁=4.5 s⁻¹ mM⁻¹ and R₂=5.5 s⁻¹ mM⁻¹, in aqueous solution at 1.5 T and are assumed to be the same in tissue. The signal enhancement can then be obtained as a function of Cₜ by dividing S(Cₜ) by S(0) to give equation [e7]:

$$E ( {C}_{t} ) = \frac{S ( {C}_{t} )}{S ( 0 )} = {ⅇ}^{- {TER}_{2} {C}_{t}} ( \frac{1 - {ⅇ}^{- TR ( {T}_{10}^{- 1} + {R}_{1} {C}_{t} )} - cos α ( {ⅇ}^{- {TRT}_{10}^{- 1}} - {ⅇ}^{- TR ( 2 {T}_{10}^{- 1} + {R}_{1} {C}_{t} )}}{1 - {ⅇ}^{- {TRT}_{10}^{- 1}} - cos α ( {ⅇ}^{- TR ( {T}_{10}^{- 1} + {R}_{1} {C}_{t} )} - {ⅇ}^{- TR ( 2 {T}_{10}^{- 1} + {R}_{1} {C}_{t} )}} )$$

By using equation e6 or e7, a calculation of the concentration of contrast agent in each voxel can be obtained from the dynamic MR data using the values of T₁ calculated by the technique above. This more accurate value for the concentration of contrast agent can then be used in a pharmacokinetic model in the derivation of certain useful and physiologically meaningful parameters relating to the tissue being imaged. Several different pharmacokinetic models have been published describing the time varying distribution of contrast agent in different “compartments” of the body. FIG. 3 illustrates a two-compartment model. The two-compartment model consists of a central compartment corresponding to the blood plasma pool, which is able to exchange, via rate constant kₚₑ and kₑₚ, with the lesion leakage space or extravascular extracellular space (EES). The initial concentration of contrast agent in the blood plasma is determined by the administered dose and is depleted by the loss of contrast agent to the kidney governed by the rate parameter kₒᵤₜ. The concentration-time curves observed in the dynamic MR imaging are assumed to result from changes in contrast agent concentration in the EES corresponding to contrast uptake by the lesion from the plasma. The solution of the pharmacokinetic model is therefore found to describe this concentration in terms of the various rate and volume parameters of the model.

The equations describing the concentration of Gd in each compartment as a function of time can be constructed by considering ‘conservation of mass’ within the model and are given by:

$$\begin{matrix}{V}_{p} \frac{ⅆ {C}_{p}}{ⅆ t} = {k}_{ep} {V}_{e} {C}_{e} - ( {k}_{pe} + {k}_{out} ) {V}_{p} {C}_{p} + {M}_{in} & [ e8 ] \\ {V}_{e} \frac{ⅆ {C}_{e}}{ⅆ t} = {k}_{ep} {V}_{p} {C}_{p} - {k}_{ep} {V}_{e} {C}_{e} & [ e9 ]\end{matrix}$$

where Mᵢₙ(t) represents the mass input function of injected Gd and Vₚ and Vₑ represent the volumes of the plasma and EES compartments, respectively. The solution to these two equations can be obtained, for example by using Laplace transforms (l), and assuming that Mᵢₙ takes the form of an impulse function (instantaneous injection) i.e. l(D/Vₚ)=D/Vₚ, the solution is:

$${C}_{t} = \frac{D {ν}_{e} {K}^{trans}}{{V}_{p} ( {K}^{trans} - {k}_{out} {ν}_{e )}} [ exp ( - {k}_{out} t ) - exp ( - \frac{{K}^{trans}}{{ν}_{e}} t ) ] \qquad [ e10 ]$$

The model is described by the two physiological parameters, the transfer coefficient K^{trans} and the rate constant kₑₚ=K^{trans}/νₑ. These two parameters characterise completely the uptake curves, assuming a normal plasma excretion rate (determined by kₒᵤₜ/Vₚ), and have a known physiological meaning whereby K^{trans} is the permeability surface area product per unit volume of tissue and νₑ is the extravascular extracellular space (EES) volume fraction.

Further pharmaco-kinetic analysis methods have been developed based on a three-compartment model, which considers the exchange between the plasma, the whole body extracellular space and the lesion leakage space or EES, as illustrated in FIG. 3(b). Again, the initial plasma concentration is determined by the administered Gd dose and is depleted by excretion to the kidneys (rate constant kₒᵤₜ). In this model, exchange occurs between the plasma and the extracellular space over the whole body and results in a biexponential decay of plasma concentration with time. Superimposed on this ‘natural’ biexponential decay is a further term that characterises the interaction between the plasma and lesion leakage space compartments. In practice, the interaction between the plasma and extracellular space is characterised by fitting to data obtained from a series of normal volunteers. They hypothesise that these fitted parameters are unlikely to vary much between normal and pathological subjects and that any small variations are likely to have little effect on the calculated volume and rate parameters, when compared to other errors such as in the measurement of T₁₀.

Conservation of mass can again be used to provide a mathematical formalism of the compartmental model. The plasma concentration can be described by considering the flow from the plasma to the extracellular space and kidneys as shown in e12below,

$$- {V}_{p} \frac{ⅆ {C}_{p}}{ⅆ t} = {k}_{px} ( {C}_{p} - {C}_{x} ) + {k}_{out} {C}_{p} \qquad [ e12 ]$$

where it is assumed that kₚₓ=kₓₚVₚ is the plasma volume and Cₚ and Cₓ are the plasma and extracellular space volumes, respectively. The constants kₚₓ and kₒᵤₜ describe the flow rate per unit concentration difference [in ml min⁻¹]. Similarly, flow in the extracellular space is given by

$${V}_{x} = \frac{ⅆ {C}_{x}}{ⅆ t} = ( {C}_{p} - {C}_{x} ) \qquad [ e13 ]$$

where Vₓ is the volume of the extracellular space. These equations can be combined, by eliminating Cₓ and the resulting differential equation solved, to give

Cₚ(t)=D(α₁e^{-m}^{₁}^{t}+α₂e^{-m}^{₂}^{t}) [e14]

Under the assumption that kₚₓ>>kₒᵤₜ and using the initial conditions of Cₚ=D/Vₚ and Cₓ=0, where D is the administered Gd dose, the following constants are obtained

$$\begin{matrix}\begin{matrix}{m}_{1} = \frac{{k}_{px} ( {V}_{p} + {V}_{x} )}{{V}_{p} {V}_{x}} & & & {m}_{2} = \frac{{k}_{out}}{{V}_{p} + {V}_{x}}\end{matrix} & [ e15 ] \\ \begin{matrix}{a}_{1} = \frac{{V}_{x}}{{V}_{p} ( {V}_{p} + {V}_{x} )} & & & & {a}_{2} = \frac{1}{{V}_{p} + {V}_{x}}\end{matrix} & [ e16 ]\end{matrix}$$

The curve obtained in e14 yielded the following values when fitted to data from normal volunteers with Gd dose of 0.25 mM kg⁻¹, α₁=3.99 kg 1⁻¹, α₂=4.78 kg 1⁻¹, m₁=0.144 min⁻¹ and m₂=0.0111 min⁻¹.

For a complete pharmacokinetic analysis the interaction between the plasma and EES (υₑVₜCₑ) must be considered where υₑ is the fraction of lesion tissue occupied by the leakage space, Vₜ is the lesion tissue volume and Cₑ is the Gd concentration within the EES. The flow of contrast agent between the compartments can then be described by

$${υ}_{d} \frac{ⅆ {C}_{e}}{ⅆ t} = {k}_{pe} ( {C}_{p} - {C}_{e} ) \qquad [ e17 ]$$

where kₚₑ=kₑₚ=PS/Vₜ, P is the permeability coefficient and S is the surface area of the leaking membrane. The transfer coefficient kₚₑ has units of min⁻¹ and can also be described as the ‘permeability surface area product per unit volume of tissue’. The Gd concentration in the lesion is then obtained by substituting e14 into e17 and solving the resulting differential equation to give

$${C}_{t} ( t ) = {Dk}_{pe} {\sum }_{i = 1}^{2} \frac{{a}_{i} ( {e}^{- {m}_{3} t} - {e}^{- {m}_{i} t} )}{{m}_{i} - {m}_{3}} \qquad [ e18 ]$$

where m₃=kₚₑ/υₑ and Cₜ=υₑ Cₑ is the total Gd concentration in the tussue and is assumed to result only from contrast agent within the EES.

The concentration-time curve is described by e18 for the three-compartment model (c.f. Eq. [e10] for the two-compartment model) and can be fitted for the two unknown parameters kₚₑ and υₑ, as before, using standard non-linear fitting routines such as the Levenberg-Marquardt method. The transfer coefficient kₚₑ gives information on the physiological coupling rate between the plasma and lesion compartments, while the volume fraction υₑ gives the relative volume of tissue occupied by the leakage space. Care is required in the interpretation of these physiological parameters, particularly regarding some of the assumptions made in their derivation. For example, it is implicitly assumed that the Gd concentration is evenly distributed within a compartment, which may not be the case in high permeability lesions, where the capillary flow may not be sufficient to maintain the plasma concentration in this local region. Thus the permeability term kₚₑ should be referred to as an apparent permeability, due its potential contamination by the flow component.

Thus using the techniques above, for each voxel in the image the values of the longitudinal relaxation time T₁, the transfer coefficient K^{trans}=kₚₑ and the extravascular extracellular space volume fraction νₑ can be obtained. As will be demonstrated by reference to the experimental results illustrated in FIGS. 4, 5, 6 and 7 and discussed below, these parameters provide a physiologically meaningful and highly useful characterisation of the tissue being imaged and are capable of distinguishing between malignant tissue and benign tissue.

Each voxel in the volume can be represented by a parameter “vector”, which describes the relevant physiological properties of the tissue. This parameter “vector” is defined to be x=[x₁x₂x₃]^{T}=[K^{trans}, kₑₚ,1/T₁]^{T}, where all parameters have units of seconds⁻¹. Maps are then produced whereby a vector in 3-D space represents each voxel in the image and the distribution of these vectors can be used to visualise the type of tissue. An effective representation is to visualise the parameter vector using colour, for example RGB, CMY, or HSB colour channels, or different textures.

In the case of the colour representation the colour indexing is normalised, for instance so that each colour channel runs from a value of 0 to a value of 1. This can be done by scaling the data to a likely ‘maximum’ based on observation (or values from the literature). The parameter is divided by this ‘maximum’ to normalise it and anything with a value greater than the ‘expected’ maximum is set to 1. In the current implementation the scaling parameters (expected maximums) for each channel are:

K^{trans}(red)−0.1 min−1

kₑₚ(green)−0.2 min−1

T₁(blue)−1500 ms

However, depending on the display method (monitor, film, paper etc.) these parameters are altered or the scaling is conducted as appropriate

The parameter vector representation enables many methods developed to analyse vector fields to be utilised in order that relevant features can be extracted from the volume data. Furthermore, a modification of the ‘local phase coherence’, which has previously been developed for analysis of magnetic resonance angiography data (see A. C. S. Chung, J. A. Noble, Fusing magnitude and phase information for vascular segmentation in phase contrast MR angiograms; Procs. Of MICCAI, pp. 166-175,2000), can be used to produce a physiologically relevant segmentation of malignant lesions. The coherence measure used compares each vector to a reference value defined at an angle θ using a normalised dot product and is therefore called the ‘relative phase coherence’. In the case of segmenting malignant tumours, a (x₁x₂) plane angle of θ=45° was used because this corresponds to the case where vνₑ=1.0 and therefore 100% of the voxel is occupied by EES and also the T₁ value is high (and therefore the vector lies largely in the x₁-x₂ plane).

FIG. 4 shows typical 2-D coronal pharmacokinetic parameter maps of Kₜᵣₐₙₛ and kₑₚ along with a map of T₁ for a patient demonstrating a typical ring enhancement that is characteristic of malignancy. FIG. 5(a) shows the RGB parameter vector representation for the same coronal slice as FIG. 4. FIG. 5(b) shows an enlargement of the tumour region with parameter vectors overlaid onto an uptake curve integral map. In this example, a 2-D visualisation is presented which demonstrates only the in-plane (x₁x₂) component and the T₁ value is encoded such that high intensity vectors represent high T₁ values and low intensity represents low values. FIG. 5(c) shows a map of the relative phase coherence (θ=45°) illustrating how this measure correctly identifies the malignant tumour tissue. The difference in phase angle between the enhancing outer region and the necrotic centre is clearly visible and is exploited in the production of the ‘relative phase coherence’ map which enhances the region of significant contrast uptake, as shown in (c).

FIG. 6 illustrates further results comparing for four patients the conventional signal enhancement based analysis (FIG. 6a) with the physiological colour representation (FIG. 6b). In the conventional signal enhancement-based image, regions of high enhancement are shown as high intensity. But there is no distinction as to whether the high enhancement occurs because of high uptake of contrast agent or high intrinsic T₁ value. In the physiological colour representation of the FIG. 6bregions of high permeability and EES volume fraction are shown as yellow/white and typically correspond to malignant lesions. Regions with high permeability, but low EES volume fraction are shown in red or magenta, and identify more benign regions. Regions which enhance simply because of their T₁ characteristics are indicated in blue, and again are suggestive of benign regions.

An important point to note in the results shown is that with the physiological colour representation the tumours are illustrated as having a bright (signal enhancing) outer ring, with a dark (non-enhancing) centre. This is interesting and demonstrates the power of the technique because tumours typically have a necrotic centre surrounded by the microvasculature. Therefore the physiological colour based representation is revealing the true physiology of the tumour. This contrasts with the conventional signal-enhancement images which do not distinguish between the necrotic centre and the microvasculature. This is because the necrotic centre enhances because it has a high T₁ value (not because it has a high uptake of contrast agent).

As well as being useful as an enhanced diagnostic aid, the technique is also useful in judging the effectiveness of the treatment, such as chemotherapy or radiotherapy. One of the main aims of such therapy is to destroy the microvasculature. Because the technique described above correctly distinguishes the microvasculature from the necrotic centre of the tumour, the success of the therapy can be judged easily and accurately. Further, the fact that chemotherapy tends to change the tissue type, which may change the T₁ value, does not confuse the technique because the T₁ value is calculated. FIG. 7 illustrates this and shows for two patients a comparison of the conventional signal enhancement analysis method and the physiological-based colour representation both before and after chemotherapy. FIGS. 7(a) and (b) relate to the results in one patient and FIGS. 7(c) and (d) in another patient. As can be seen in FIG. 7(a), the conventional image, while a comparison of the pre-chemo and post-chemo images demonstrate that the therapy is working to an extent, a tumour is still indicated as being present, though shrunk, in one breast after treatment. However, the physiological based colour representation of FIG. 7(b) reveals that actually the bright ring of microvasculature has completely disappeared post-chemotherapy, suggesting that little malignant tissue remain. This was supported by the histological assessment for the excised lesion, which found only localised fibrosis and no residual malignancy.

The accuracy of the technique also allows the images to be used in the planning of surgical intervention because the true extent of malignant tissue is revealed by these techniques, and thus the unnecessary removal of non-malignant tissue can be avoided.

While the above embodiment has been described with particular reference to breast cancer imaging, the invention is applicable to imaging of other soft tissues, including organs such as the brain or prostate etc. Further, the techniques are applicable to other imaging pulse sequences on other types of apparatus and using other types of contrast agent.

1: A method of enhancing a dynamic contrast-enhanced magnetic resonance image comprising the steps of:
for each voxel of the image fitting to the magnetic resonance signal a parameterised pharmaco-kinetic model of the contrast enhancement process in the sample being imaged to calculate the values of parameters of the model which represent properties of the imaged sample,
and displaying the image with each of said parameters being represented in a visually distinguishable manner.

2: A method according to claim 1 wherein the parameters are each represented by a different colour whose intensity is representative of the value of the parameter.

3: A method according to claim 1 wherein the parameters for each of a plurality of regions of the sample are represented as components of a vector displayed for each region.

4: A method according to claim 3 wherein at least one of the parameters is represented by the intensity or colour of the displayed vector.

5: A method according to claim 1 wherein the parameters are represented in a relative phase coherence map.

6: A method according to claim 1 wherein the parameters include at least one parameter representative of the physiology of the imaged sample.

7: A method according to claim 6 wherein the at least one parameter representative of the physiology of the imaged sample is at least one of: the extravascular extracellular space (EES) volume fraction, and the permeability surface area product per unit volume of the sample (K^{trans}).

8: A method according to claim 1 wherein the parameters include at least one parameter representative of the structure of the imaged sample.

9: A method according to claim 8 wherein the at least one parameter representative of the structure of the imaged sample is the longitudinal relaxation time (T₁).

10: A method according to claim 1 wherein the parameterised pharmaco-kinetic model is a two- or three-compartment pharmaco-kinetic model.

11: A method of magnetic resonance imaging comprising the steps of:
acquiring resonance signals by applying to a subject successive electromagnetic pulse sequences, each sequence differing in a selected acquisition parameter,
and calculating from the resonance signals the longitudinal relaxation time (T₁) for the sample.

12: A method according to claim 11 wherein the selected acquisition parameter which differs from sequence to sequence is the flip angle.

13: A method according to claim 11 wherein the selected acquisition parameter which differs from sequence to sequence is the repetition time (TR).

14: A method according to claim 12 wherein the selected acquisition parameter is varied from sequence to sequence to minimise the error in the longitudinal relaxation time (T₁) over the range expected in the sample, such as, by example the Monte Carlo simulation/method.

15: A method according to claim 11 wherein the pulse sequence is a gradient echo sequence.

16: A method according to claim 15 wherein the pulse sequence is a T₁ weighted 3D fast spoiled gradient echo sequence.

17: A method according to claim 11 wherein the longitudinal relaxation time (T₁) is calculated by fitting the resonance signals for the successive sequences to a model of the sample's response to the pulse sequence.

18: A method according to claim 17 wherein the model includes correction for non-uniform excitation across the sample.

19: A method according to claim 17 wherein the model includes correction for bias field (B₁) inhomogeneity across the sample.

20: A method according to claim 11 wherein the longitudinal relaxation time (T₁) is calculated for each voxel of the sample.

21: A method according to claim 11, further comprising applying a contrast agent and further electromagnetic pulse sequences to the sample to produce a dynamic contrast-enhanced magnetic resonance image, and enhancing the image by the steps of:
for each voxel of the image fitting to the magnetic resonance signal a parameterised pharmaco-kinetic model of the contrast enhancement process in the sample being imaged to calculate the values of parameters of the model which represent properties of the imaged sample,
and displaying the image with each of said parameters being represented in a visually distinguishable manner.

22: A method according to claim 1 wherein the sample is soft tissue in the human or animal body.

23: A method according to claim 1 wherein the sample is a human breast.

24: Magnetic resonance imaging apparatus comprising a data processor and a display, the data processor being adapted to calculate the values of parameters of the model which represent properties of the imaged sample, and the display being operable to display the parameters, in accordance with the method of claim 1.

25: Magnetic resonance imaging apparatus according to claim 24 further comprising a controller for causing the application to the sample of successive electromagnetic pulse sequences, each sequence having a different flip angle, the data processor being adapted to calculate from the resonance signals the longitudinal relaxation time (T₁) for the sample, by the steps of:
acquiring resonance signals by applying to a subject successive electromagnetic pulse sequences, each sequence differing in a selected acquisition parameter,
and calculating from the resonance signals the longitudinal relaxation time (T₁) for the sample.

26: A computer program comprising program code means for executing on programmed data processing apparatus according to the method of claim 1.
